(19) United States
(12) Patent Application Publication
(10) Pub. No.: US 2023/0099972 A1
(43) Pub. Date: Mar. 30, 2023

(54) COMPOSITIONS AND METHODS FOR TREATING HEADACHES

(71) Applicant: ATAI Life Sciences AG, Berlin (DE)

(72) Inventors: Srinivas G. RAO, Encinitas, CA (US); Glenn SHORT, Scituate, MA (US); Carrie BOWEN, Uxbridge, MA (US)

(21) Appl. No.: 17/957,851

(22) Filed: Sep. 30, 2022

Related U.S. Application Data
(60) Provisional application No. 63/250,595, filed on Sep. 30, 2021.

(51) Int. Cl. A61K 31/4045; A61P 25/06

(52) U.S. Cl. CPC A61K 31/4045; A61P 25/06

(57) ABSTRACT

The present disclosure provides new methods of treating headache disorders with 5-methoxytryptamine or 5-hydroxytryptamine and compositions thereof

### CROSS-REFERENCE TO RELATED APPLICATION

The present application claims the benefit of priority to U.S. Provisional Application No. 63/250,595, filed Sep. 30, 2021, the contents of which are hereby incorporated by reference in its entirety.

### BACKGROUND OF THE INVENTION

### (i) Field of the Invention

The present invention relates to compositions and methods for treating headaches and, more particularly, to treatment of acute headaches in a patient using certain tryptamine compounds as well as prodrugs or salts thereof.

### (ii) Description of Related Art

Migraine and cluster headache are episodic and often chronic headache disorders that can lead to significant disability (Andersson et al., 2017; Harriott et al., 2019). Migraine has a worldwide prevalence of nearly 15% and is associated with severe recurrent headaches with accompanying sensitivity to light and/or sound, nausea and vomiting. The condition is heterogeneous and involves both genetic and environmental factors. Migraine pain is mediated by activation of trigeminal nociceptors innervating meningeal tissues, including dural arteries and sinuses, and release of neuropeptides, such as calcitonin gene related peptide (CGRP), substance P, neurokinin A and/or pituitary adenylate cyclase activating peptide (PACAP). Additional brain areas, such as the cortex, thalamus and hypothalamus, also are believed to be involved in the generation of a migraine episode (Puledda et al., 2017). Cluster headache is a rarer, sometimes treatment-resistant and particularly painful condition with a prevalence around 1 in 1000 individuals and without any pharmacological treatments specifically developed for this disorder. There is a need in the art for new methods of treating headaches.

Acute pharmacological treatment of migraine may include ergotamine, triptans, CGRP receptor antagonists (Gepants) or 5-HT1F receptor agonists (ditans) (Puledda et al., 2017). Ergotamine is a non-hallucinogenic ergot alkaloid originally isolated from fungus with anti-migraine activity that is believed to be mediated by agonism at serotonin 5-HT1B/1D receptors, but its lack of pharmacological selectivity leads to unwanted side effects (Yu, 2008). Triptans, such as sumatriptan, are selective serotonin 5-HT1B/1D receptor agonists that modify release of neurotransmitters from the central terminals of trigeminal primary afferent fibers, are clinically useful migraine-abortive drugs with improved tolerability over ergotamine, and also are prescribed for the treatment of cluster headache. However, side effects (e.g., cardiovascular) limit the use of triptans, and lack of efficacy or recurrence of migraine symptoms has been seen in over 50% of cases (Puledda et al., 2017). Gepants, such as ubrogepant which was approved in 2019, are effective acute migraine treatments with improved tolerability over triptans, but some compounds in this class have been associated with liver toxicity (Puledda et al., 2017). Similarly, the ditans, such as lasmiditan which was approved by the FDA in 2019, exhibit acute anti-migraine activity without cardiovascular effects, although mild central nervous system side effects have been reported (Puledda et al., 2017).

Lack of efficacy and adverse effects of available medications have prompted the search for alternative treatments for acute headache disorders. A few studies and anecdotal reports indicate that psychoactive tryptamines, a structural class of compounds containing an indolealkylamine backbone, are effective against migraine and cluster headache, often at sub-psychoactive doses and with long-lasting effects. Andersson et al. (2017) reported the following tryptamines as being specifically discussed in online forums for the alleviation of headache disorders: psilocybin, LSD, 1P-LSD, AL-LAD, D-Lysergic acid amide, DMT, 4-AcO-MET, 4-AcO-DMT, 4-HO-MiPT, 4-HO-MET, 5-MeO-MiPT and 5-MeO-DALT. The non-psychoactive LSD analog, 2-bromo-LSD or BOL-148, also has shown a promising efficacy versus side effect profile for the treatment of cluster headache. Madsen et al. 2022 reports that three doses of psilocybin, each separated by seven days, prophylactically reduced chronic cluster headache attack frequency in a 10-patient open-label study (Madsen et al., 2022). One of these 10 patients experienced complete remission for 21 weeks, beginning one day after the first psilocybin session. These indole alkaloid compounds interact with serotonin receptors, including 5-HT2A, 5-HT1B and/or 5-HT1D, and have structural similarity to the triptans.

Dimethyltryptamine (DMT) and its analogs belong to a class of drugs referred to as psychedelics (“mind-manifesting” drugs). Specifically, DMT and analogs are considered 5-hydroxytryptaminergic (serotonergic) psychedelics, like other tryptamines such as psilocybin, ergolines such as lysergic acid diethylamide (LSD). DMT is a naturally occurring alkaloid found in animal and plant species. Like DMT, the analogs 5-methoxydimethyltryptamine (5-MeO-DMT) and 5-hydroxydimethyltryptamine (5-OH-DMT, bufotenine) are naturally occurring alkaloids found in some toad and plant species. There remains a need in the art for improved compositions and methods for treating patients using DMT and its analogs.

### SUMMARY OF THE DISCLOSURE

In one aspect, the present disclosure provides methods of treating a headache disorder by administering a therapeutically effective amount of a tryptamine to a patient in need thereof. In embodiments, the tryptamine is 5-methoxy-N,N-dimethyltryptamine (5-MeO-DMT) or a prodrug or a pharmaceutically acceptable salt thereof, or 5-hydroxy-N,N-dimethyltryptamine (5-OH-DMT) or a prodrug or a pharmaceutically acceptable salt thereof.

In embodiments, the disclosure provides methods of treating an acute headache in a patient in need thereof, the method comprising: administering a therapeutically effective amount of a tryptamine to the patient, wherein the tryptamine is 5-methoxy-N,N-dimethyltryptamine (5-MeO-DMT) or a prodrug or a pharmaceutically acceptable salt thereof, or 5-hydroxy-N,N-dimethyltryptamine (5-OH-DMT) or a prodrug or a pharmaceutically acceptable salt thereof.

In embodiments, the acute headache is an acute migraine or a cluster headache episode.

In embodiments, the tryptamine is 5-MeO-DMT or a prodrug or a pharmaceutically acceptable salt thereof. In embodiments, the method comprises administering about 0.25 mg to about 50 mg of 5-MeO-DMT to a patient in need thereof. In embodiments, about 0.25 mg to about 36 mg of 5-MeO-DMT is administered to a patient in need thereof.

In embodiments, the tryptamine is 5-OH-DMT or a prodrug or a pharmaceutically acceptable salt thereof. In embodiments, about 0.25 mg to about 50 mg of 5-OH-DMT is administered to a patient in need thereof.

In embodiments, the tryptamine is administered by insufflation, intranasally, orally, subcutaneously, sublingually, buccally, or by inhalation.

In embodiments, the method comprises administering a single dose of the tryptamine to the patient. In embodiments, the method further comprises administering a second dose tryptamine to the patient about 30 minutes after the first administration, wherein the second dose is administered if the first dose is not therapeutically effective. In embodiments, the tryptamine is administered at the onset of the acute headache. In embodiments, the administration reduces the patient's pain associated with the acute headache.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”,“about 50” means a range extending to less than half the interval(s) between the preceding and subsequent values, e.g., more than 49.5 to less than 52.5.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound, or prodrug thereof, or a salt thereof, that, when administered to a patient, is capable of achieving the intended result. For example, an effective amount of a salt of 5-OH-DMT is that amount that is required to reduce at least one symptom of headache in a patient. The actual amount that comprises the “effective amount” or “therapeutically effective amount” will vary depending on a number of conditions including, but not limited to, the severity of the disorder, the size and health of the patient, and the route of administration.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “prodrug” as used herein refers to a derivative of the compound (e.g., 5-MeO-DMT or 5-OH-DMT) containing a functional group (such as an ester or amide) that is capable of releasing the compound when the prodrug form is administered to a patient. In embodiments, the functional group that releases 5-MeO-DMT from the 5-MeO-DMT prodrug is attached to the indole nitrogen atom of 5-MeO-DMT. In embodiments, the functional group that releases 5-OH-DMT from the 5-OH-DMT prodrug is attached to the indole nitrogen atom of 5-OH-DMT. In embodiments, the functional group that releases 5-OH-DMT from the 5-OH-DMT prodrug is attached to the phenolic oxygen atom of 5-OH-DMT. In embodiments, the 5-MeO-DMT prodrug or 5-OH-DMT prodrug is a compound as described in U.S. Provisional Application Ser. Nos. 63/208,874, 63/229,879, and U.S. patent application Ser. No. 17/836,984 which are hereby incorporated by reference in their entireties.

The term “salts” as used herein embraces pharmaceutically acceptable salts commonly used to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid.

The term “treating” as used herein with regard to a patient, refers to improving at least one symptom of the patient's headache (for example, headache). Treating can be improving, or at least partially ameliorating a disorder.

As used herein, “therapeutically-effective dose” means a dose sufficient to achieve the intended therapeutic purpose, such as, to alleviate a sign or symptom of a disease or disorder in a patient.

The term “therapeutic effect” as used herein refers to a desired or beneficial effect provided by the method and/or the composition. For example, the method for treating headache provides a therapeutic effect when the method reduces at least one symptom of headache in a patient.

### DETAILED DESCRIPTION OF PREFERRED EMBODIMENTS

### 5-MeO-DMT and 5-OH-DMT

The present disclosure provides method of treating headache disorders by administering 5-methoxy-dimethyltryptamine (5-MeO-DMT) or a prodrug or a pharmaceutically acceptable salt thereof or 5-hydroxy-dimethyltryptamine (5-OH-DMT) or a prodrug or a pharmaceutically acceptable salt thereof.

5-Methoxy-dimethyltryptamine (5-MeO-DMT) is a natural psychoactive indolealkylamine found in toad venom of Bufo alvarius and a number of plants. The compound has been used historically by indigenous cultures and produces a range of rapid and short-lasting subjective effects, including altered visual, auditory and time perceptions, euphoria and interconnectedness, as well as physical effects when consumed by smoking or the intravenous route (Shulgin and Shulgin, 1997; Ott, 2001b; Barsuglia et al., 2018; Uthaug et al., 2020). Inhaled 5-MeO-DMT administered in a naturalistic group setting to individuals using the compound for spiritual purposes was associated with improvements in self-reported symptoms of depression and anxiety (Davis et al., 2019). Interestingly, a recent human field study reported that single inhalation of vapor from dried toad secretion containing 5-MeO-DMT (and very low amounts of its active metabolite, 5-OH-DMT) produced improvements in affect and cognition at 24 hours, which was maintained at 4 weeks following administration (Uthaug et al., 2019). In its Form F-1 filing, GH Research PLC reported completion of a single-dose Phase 1 clinical trial of their proprietary inhaled 5-MeO-DMT preparation (GH001) at 12 and 18 mg in 16 patients with treatment-resistant depression, which found clinical remission in 2 patients on day 7 after receiving 12 mg and 1 patient on day 7 after receiving 18 mg and a positive clinical response in 1 patient on day 7 after receiving 18 mg (https://sec.report/Document/0001140361-21-019900/). Apparent rapid onset and maintained therapeutic-like effects following single administration also have been reported for other psychedelic drugs, such as psilocybin to produce clinical antimigraine and antidepressant effects and MDMA to produce clinical anxiolytic efficacy (Schindler et al., 2020; Carhart-Harris et al., 2017; Mithoefer et al., 2010).

5-MeO-DMT is inactivated mainly by monoamine oxidase A and, to a more limited extent, O-demethylated by CYP2D6 to an active metabolite, 5-OH-DMT or bufotenine (Shen et al., 2010). 5-OH-DMT also is found naturally in toads and plants. Reports of its rapid and short-lived psychoactivity include altered visual perceptions and physical effects, such as body area tightness, facial flushing and nausea, when consumed intravenously, intranasally or sublingually (Shulgin and Shulgin, 1997; McBride, 2000; Ott, 2001a).

5-MeO-DMT is a potent agonist across many serotonin (5-HT) receptor subtypes (e.g., 5-HT1A, 1B, 2A, 2B, 6) and also binds to multiple dopaminergic and noradrenergic alpha2 receptor subtypes, serotonin and norepinephrine transporters, and imidazolinel receptors (Ray, 2010; Cameron et al., 2021). 5-MeO-DMT induces the head twitch response in mice, indicative of in vivo 5-HT2A receptor activation, while agonism at 5-HT1A receptors also is involved in its behavioral effects in animals (Winter et al., 1999; Halberstadt et al., 2011; Cameron et al., 2021; Dunlap et al., 2020). 5-MeO-DMT stimulates neuroplastic changes, as evidenced by increased neurite outgrowth in cortical cultures, altered protein expression in human cerebral organoids, and enhanced proliferation and maturation of hippocampal dentate gyrus granule cells in adult mice (Dunlap et al., 2020; Dakic et al., 2017; Lima da Cruz et al., 2018). Of potential relevance to the described method of treatment of a painful condition, such as headache, 5-MeO-DMT exhibited acute analgesic effects in rats (Archer et al., 1986). The active metabolite 5-OH-DMT also binds to multiple subtypes of 5-HT receptors, including binding and stimulation of 5-HT2A receptor signaling with increased potency relative to 5-MeO-DMT (Lyon et al., 1988; Roth et al., 1997; McBride, 2000). 5-OH-DMT induced the head twitch response in mice (ED50=15 mg/kg, subcutaneous), indicative of in vivo 5-HT2A receptor activation (Come and Pickering, 1967). In addition, 5-OH-DMT exhibited acute anti-inflammatory and analgesic effects in mice (Wang et al., 2021).

5-MeO-DMT has reported psychoactive effects in humans in the range of 6-30 mg when smoked, 0.25-3.1 mg when administered intravenously, or ˜10 mg sublingually (TiHKAL, Shulgin & Shulgin, 1997; Shen et al., 2010). Upon insufflation of 10 mg 5-MeO-DMT, initial effects were reported at 3-4 minutes, peaking at 35-40 minutes and terminating by 60-70 minutes (Ott, 2001b). There are reports of psychoactive effects following oral administration (activity at 10-25 mg following pretreatment with a monoamine oxidase inhibitor (e.g., harmaline) or 30 mg when encapsulated; TiHKAL, Shulgin & Shulgin, 1997; Ott, 2001b), but the reduced potency and anticipated slower pharmacokinetic profile of the oral route would not be preferred for the treatment of acute headache. Similarly, 5-OH-DMT induces psychoactive effects in humans following administration via inhalation of vapor, intranasally and sublingually, with lower potency following the oral (encapsulated) route (Ott, 2001a). Upon insufflation of 40 mg 5-OH-DMT, initial effects were reported at 5 minutes, peaking at 35-40 minutes with short-lived after effects for up to 90 minutes (Ott, 2001a).

### Formulations

The methods of the present invention can employ various formulations for administration to patients, e.g., humans in unit dosage forms, such as tablets, capsules, pills, powders, granules, sterile parenteral solutions or suspensions (e.g., intramuscular (IM), subcutaneous (SC) and intravenous (IV)), transdermal patches, and oral solutions or suspensions, and oil-water emulsions containing suitable quantities of a tryptamine as described herein.

Oral pharmaceutical dosage forms can be either solid or liquid. The solid dosage forms can be tablets, capsules, granules, films (e.g., buccal films) and bulk powders. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art. In some embodiments, the present oral dosage forms may include orally disintegrating tablets.

In embodiments, the disclosure provides a pharmaceutical composition comprising a tryptamine and one or more pharmaceutically acceptable carriers or excipients.

In embodiments, the pharmaceutical composition comprises from about 0.1 mg to about 100 mg of tryptamine. In embodiments, the pharmaceutical composition comprises from about 2 mg to about 40 mg, from about 2 mg to about 10 mg, from about 5 mg to about 30 mg, from about 5 mg to about 15 mg, or from about 20 mg to about 30 mg of a tryptamine. In embodiments, the pharmaceutical composition comprises about 1 mg, about 5 mg, about 10 mg, or about 25 mg of the tryptamine.

In embodiments, the pharmaceutical composition comprises 5-MeO-DMT. In embodiments, the pharmaceutical composition comprises 5-OH-DMT.

### METHODS OF THE DISCLOSURE

In one aspect, the present disclosure provides methods of treating a headache disorder by administering a therapeutically effective amount of a tryptamine to a patient in need thereof. In embodiments, the tryptamine is 5-methoxy-N,N-dimethyltryptamine (5-MeO-DMT) or a prodrug or a pharmaceutically acceptable salt thereof, or 5-hydroxy-N,N-dimethyltryptamine (5-OH-DMT) or a prodrug or a pharmaceutically acceptable salt thereof. In embodiments, the tryptamine is administered in a pharmaceutical composition as described herein.

In embodiments, the methods of the present disclosure are used to treat headache disorders including migraines, classic migraines, migraine disorders, headaches, chronic migraines, migraines with aura, episodic migraines, acute migraines, vestibular migraines, tension type headaches, medication overuse headaches, menstrual migraines, cluster headaches, chronic headaches, post traumatic headaches, postdural puncture headaches, chronic cluster headaches, and episodic cluster headaches.

In embodiments, the present disclosure provides a method of treating an acute headache in a patient in need thereof, the method comprising: administering a therapeutically effective amount of a tryptamine to a patient in need thereof, wherein the tryptamine is 5-methoxy-N,N-dimethyltryptamine (5-MeO-DMT) or a prodrug or a pharmaceutically acceptable salt thereof, or 5-hydroxy-N,N-dimethyltryptamine (5-OH-DMT) or a prodrug or a pharmaceutically acceptable salt thereof.

In embodiments, the acute headache is an acute migraine. In embodiments, the acute headache is a cluster headache episode.

In embodiments, the present disclosure provides methods of treatment of an acute single migraine and/or cluster headache episode by administering a single dose of 5-MeO-DMT and/or 5-OH-DMT to a patient in need thereof.

According to the methods of the present disclosure, the tryptamine (e.g., 5-MeO-DMT) may be administered to the patient in need thereof using any suitable route of administration known to those skilled in the art, including oral, parenteral (e.g., intravenous, subcutaneous, intradermal, intramuscular, intradermal, intrapleural, intracerebral, and intra-articular), topical (e.g., to both skin and mucosal surfaces, including airway surfaces, and transdermal administration), inhalation (e.g., via an aerosol), rectal, transmucosal, intranasal, buccal, sublingual, vaginal, intrathecal, intraocular, and transdermal.

In embodiments, the tryptamine is administered by insufflation. In embodiments, the tryptamine is administered by inhalation. In embodiments, the tryptamine is intranasally administered. In embodiments, the tryptamine is orally administered. In embodiments, the tryptamine is subcutaneously administered. In embodiments, the tryptamine is sublingually administered. In embodiments, the tryptamine is buccally administered.

### Dosing

In embodiments, the methods of the present disclosure comprise administering about 0.1 mg to about 100 mg, about 1 mg to about 50 mg, or about 5 mg to about 30 mg of a tryptamine (e.g., 5-MeO-DMT or 5-OH-DMT) to a patient in need thereof. In embodiments, the methods of the present disclosure comprise administering about 0.10 mg, about 0.15 mg, about 0.20 mg, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg of a tryptamine (e.g. 5-MeO-DMT) to a patient in need thereof.

In embodiments, the method of the present disclosure comprises administering a therapeutically effective amount of 5-MeO-DMT or a prodrug or a pharmaceutically acceptable salt thereof to a patient in need thereof.

In embodiments, about 0.25 mg to about 50 mg of 5-MeO-DMT is administered to a patient in need thereof. In embodiments, about 6 mg to about 30 mg of 5-MeO-DMT is administered to a patient in need thereof. In embodiments, about 10 mg of 5-MeO-DMT is administered to a patient in need thereof. In embodiments, about 0.25 mg to about 3 mg of 5-MeO-DMT is administered to a patient in need thereof. In embodiments, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg or about 50 mg of 5-MeO-DMT is administered to a patient in need thereof.

In embodiments, the method of the present disclosure comprises administering a therapeutically effective amount of 5-OH-DMT or a prodrug or a pharmaceutically acceptable salt thereof to a patient in need thereof.

In embodiments, about 0.25 mg to about 50 mg of 5-OH-DMT is administered to a patient in need thereof. In embodiments, about 6 mg to about 30 mg of 5-OH-DMT is administered to a patient in need thereof. In embodiments, about 10 mg of 5-OH-DMT is administered to a patient in need thereof. In embodiments, about 0.25 mg to about 3 mg of 5-OH-DMT is administered to a patient in need thereof. In embodiments, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg or about 50 mg is administered to a patient in need thereof.

In embodiments, the dosing frequency and dose amount per administration of the tryptamine (e.g., 5-MeO-DMT or 5-OH-DMT) are selected to provide therapeutic effects for the treatment of an acute headache.

In embodiments, the dosing frequency and dose amount per administration of the tryptamine (e.g., 5-MeO-DMT or 5-OH-DMT) are selected to provide a reduction in incidence of headaches.

In embodiments, the dosing frequency and dose amount per administration of the tryptamine (e.g., 5-MeO-DMT or 5-OH-DMT) are selected to provide a reduction in the patient's pain associated with the acute headache.

In embodiments, the dosing frequency and dose amount per administration of the tryptamine (e.g., 5-MeO-DMT or 5-OH-DMT) are selected to provide one or more of the following: improved safety outcome, decreased adverse events, reduced headache frequency, reduced number of days with headache, reduced number of participants with adverse events, improved quality of life, improvement in headache impact test, reduction in headache intensity, improvement in migraine disability assessment, reduction in headache, reduction in migraine days, reduced use of rescue medication, reduced frequency of migraine attacks, reduction in headache severity, and improved patient satisfaction.

In embodiments, the tryptamine is administered at the onset of the acute headache.

In embodiments, the tryptamine is administered in a single dose to a patient in need thereof.

In embodiments, the tryptamine is administered in multiple doses to a patient in need thereof. In some embodiments, the tryptamine is administered multiple doses at intervals that are selected to provide therapeutic effectiveness. In embodiments, a second dose tryptamine is administered to the patient about 30 minutes after the first administration, wherein the second dose is administered if the first dose is not therapeutically effective.

In embodiments, the same dose of a tryptamine is administered to a patient at each administration (e.g., 10 mg of 5-MeO-DMT is administered twice to a patient in need thereof). In embodiments, a different dose of a tryptamine is administered to a patient at each administration. In embodiments, the dose of a tryptamine administered to the patient is increased over time (i.e., dose escalation). In embodiments, the dose of a tryptamine administered to the patient is decreased over time (i.e., dose de-escalation).

### NUMBERED EMBODIMENTS OF THE DISCLOSURE

In addition to the disclosure above, the Examples below, and the appended claims, the disclosure sets forth the following numbered embodiments.

1. A method of treating an acute headache in a patient in need thereof comprising administering a therapeutically effective amount of a tryptamine comprising 5-methoxy-N,N-dimethyltryptamine (5-MeO-DMT), 5-hydroxy-N,N-dimethyltryptamine (5-OH-DMT) or prodrugs or pharmaceutically acceptable salts thereof.
2. The method of embodiment 1, wherein the acute headache is an acute migraine.
3. The method of embodiment 1, wherein the acute headache is a cluster headache episode.
4. The method of any of embodiments 1-3, comprising administering a therapeutically effective amount of 5-MeO-DMT or a prodrug or a pharmaceutically acceptable salt thereof to the patient.
5. The method of any of embodiments 1-4, comprising administering between about 0.25 mg to about 50 mg of 5-MeO-DMT.
6. The method of any of embodiments 1-5, comprising administering between about 0.25 mg to about 36 mg of 5-MeO-DMT.
7. The method of any of embodiments 1-6, comprising administering between about 6 mg to about 30 mg of 5-MeO-DMT.
8. The method of any of embodiments 1-7, comprising administering between about 0.25 mg to about 3 mg of 5-MeO-DMT.
9. The method of any of embodiments 1-7, comprising administering about 10 mg of 5-MeO-DMT.
10. The method of any of embodiments 1-3, comprising administering a therapeutically effective amount of 5-OH-DMT or a prodrug or a pharmaceutically acceptable salt thereof to the patient.
11. The method of any of embodiments 1-3 or 10, comprising administering between about 0.25 mg to about 50 mg of 5-OH-DMT.
12. The method of any of embodiments 1-11, comprising administering the tryptamine by insufflation.
13. The method of any of embodiments 1-11, comprising administering the tryptamine intranasally.
14. The method of any of embodiments 1-11, comprising administering the tryptamine orally.
15. The method of any of embodiments 1-11, comprising administering the tryptamine subcutaneously.
16. The method of any of embodiments 1-11, comprising administering the tryptamine sublingually.
17. The method of any of embodiments 1-11, comprising administering the tryptamine buccally.
18. The method of any of embodiments 1-11, comprising administering the tryptamine by inhalation.
19. The method of any of embodiments 1-18, comprising administering a single dose of the tryptamine to the patient.
20. The method of embodiment 19, further comprising administering a second dose of tryptamine to the patient about 30 minutes after a first administration if the first dose is not therapeutically effective.
21. The method of any of embodiments 1-20, comprising administering the tryptamine at the onset of the acute headache.
22. The method of any of embodiments 1-21, wherein the administration reduces the patient's pain associated with the acute headache.

### EXAMPLES

### Example 1

5-MeO-DMT and/or 5-OH-DMT will be evaluated in one or more rodent models of headache, for example:
inhibition of rat trigeminovascular protein extravasation (neuroinflammatory response) upon electrical stimulation of the trigeminal nerve;
inhibition of rat dural-evoked trigeminovascular nociceptive transmission;
inhibition of ex vivo KCl-stimulated CGRP release from the mouse trigeminovascular system;
inhibition of capsaicin (IV)- or periarterial electrical stimulation-mediated dural artery vasodilation; or
inhibition of induction of c-fos in rat nociceptive neurons located in cervical, spinal substantia gelatinosa and brainstem nucleus caudalis.

Studies may include electrophysiological recordings from convergent wide-dynamic range neurons in trigeminal nucleus caudalis (TNC) following: a) infusion of glyceryl trinitrate (GTN), b) inflammatory soup applied to brain dura matter or c) 1% capsaicin applied to brain dura matter. Acute effects of vehicle, sumatriptan positive reference, or test article on spontaneous neuron activity and/or facilitation of mechanically-evoked neuron activity will be evaluated.

### Rodent Behavioral Study

A rodent behavioral study will investigate acute 5-MeO-DMT and/or 5-OH-DMT effects on periorbital hypersensitivity following infusion of inflammatory mediators. Male Sprague-Dawley rats will be cannulated to receive supradural infusions of an inflammatory soup containing histamine, bradykinin, serotonin and prostaglandin E2 under brief anesthesia once per day over 5 consecutive days. After the 5-day inflammatory soup sensitization period, rats demonstrating facial hypersensitivity will have a one-week wash-out period and then will be challenged with 0.1 mg/kg GTN, followed by acute vehicle, sumatriptan positive reference, or test article administration (e.g., 5-MeO-DMT and/or 5-OH-DMT), with at least a one-week washout between each test condition. The design is within subjects with the same animals receiving multiple treatments in a cross-over fashion (n=12/treatment). A blinded investigator will test periorbital mechanical sensitivity using von Frey filaments with reproducible calibrated buckling forces varying from 0.4-10 g utilizing the simplified up-and-down method before drug administration and 30 min, 1.5 h, and at 2.5 h after drug administration.

A positive response is characterized by several behavioral criteria: stroking the face with a forepaw, head withdrawal from the stimulus, and head shaking. Data will be expressed as the average ±SEM and analyzed by two-way ANOVA followed by an appropriate post-hoc test to determine differences between treatment and control (Veh/GTN control group).

### In Vitro Testing

The role of 5-MeO-DMT and 5-OH-DMT's 5-HT2A receptor agonism and associated psychoactive effects in anti-migraine activity will be investigated. In vitro testing will include ranges to encompass 5-HT2A inactive and agonist concentrations (e.g., 5-MeO-DMT agonism at human 5-HT2AR EC50=1.85 nM, Emax=82% of 5-HT control, Cameron et al., 2021; 5-OH-DMT in vitro 5-HT2AR functional activity may require further characterization).

### In Vivo Testing

In vivo testing will include doses within the anticipated non-psychoactive and psychoactive ranges (e.g., 5-MeO-DMT mouse head-twitch response elicited at doses of 10 mg/kg i.p. or s.c. and higher, Halberstadt et al, 2011, Dunlap et al., 2020; 5-OH-DMT mouse head-twitch response ED50=15 mg/kg s.c., Come and Pickering, 1967, may require confirmation). 5-OH-DMT may be evaluated in in vitro binding assays across a broad panel of targets to further characterize its pharmacological effects.

### Cardiovascular Safety Evaluation of 5-MeO-DMT

Cardiovascular safety evaluation of 5-MeO-DMT and/or 5-OH-DMT may include: confirming a lack of vasoconstrictive effects using, for example, ex vivo preparations of rabbit saphenous vein, dog coronary or carotid artery, or human proximal or distal coronary artery, meningeal or cerebral arteries.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A method of treating an acute headache in a patient in need thereof comprising administering a therapeutically effective amount of a tryptamine comprising 5-methoxy-N,N-dimethyltryptamine (5-MeO-DMT), 5-hydroxy-N,N-dimethyltryptamine (5-OH-DMT) or prodrugs or pharmaceutically acceptable salts thereof.

2. The method of claim 1, wherein the acute headache is an acute migraine.

3. The method of claim 1, wherein the acute headache is a cluster headache episode.

4. The method of claim 1, comprising administering a therapeutically effective amount of 5-MeO-DMT or a prodrug or a pharmaceutically acceptable salt thereof to the patient.

5. The method of claim 4, comprising administering between about 0.25 mg to about 50 mg of 5-MeO-DMT.

6. The method of claim 4, comprising administering between about 0.25 mg to about 36 mg of 5-MeO-DMT.

7. The method of claim 4, comprising administering between about 6 mg to about 30 mg of 5-MeO-DMT.

8. The method of claim 4, comprising administering between about 0.25 mg to about 3 mg of 5-MeO-DMT.

9. The method of claim 4, comprising administering about 10 mg of 5-MeO-DMT.

10. The method of any claim 1, comprising administering a therapeutically effective amount of 5-OH-DMT or a prodrug or a pharmaceutically acceptable salt thereof to the patient.

11. The method of claim 10, comprising administering between about 0.25 mg to about 50 mg of 5-OH-DMT.

12. The method of claim 1, comprising administering the tryptamine by insufflation.

13. The method of claim 1, comprising administering the tryptamine intranasally.

14. The method of claim 1, comprising administering the tryptamine orally.

15. The method of claim 1, comprising administering the tryptamine subcutaneously.

16. The method of claim 1, comprising administering the tryptamine sublingually.

17. The method of claim 1, comprising administering the tryptamine buccally.

18. The method of claim 1, comprising administering the tryptamine by inhalation.

19. The method of claim 1, comprising administering a single dose of the tryptamine to the patient.

20. The method of claim 19, further comprising administering a second dose of tryptamine to the patient about 30 minutes after a first administration if the first dose is not therapeutically effective.

21. The method of claim 1, comprising administering the tryptamine at the onset of the acute headache.

22. The method of claim 1, wherein the administration reduces the patient's pain associated with the acute headache.
